(19) United States
(12) Plant Patent Application Publication
(10) Pub. No.: US 2004/0025209 P1
(43) Pub. Date: Feb. 5, 2004

(54) Miniature rose variety 'Poulmona'

(76) Inventors: L. Pernille Olesen, Fredensborg (DK); Mogens N. Olesen, Fredensborg (DK)

Correspondence Address: POULSEN ROSER APS, 620 S. FRONT STREET, CENTRAL POINT, OR, 97502, US

(21) Appl. No.: 10/211,119

(22) Filed: Aug. 2, 2002

(51) Int. Cl. A01H 5/00

(52) U.S. Cl. PLT/116

(57) ABSTRACT

A new miniature rose which has large, deep pink flowers and attractive foliage. The variety successfully propagates from softwood cuttings and is suitable for year round production in commercial glasshouses. This new and distinct variety has shown to be uniform and stable in the resulting generations from asexual propagation.

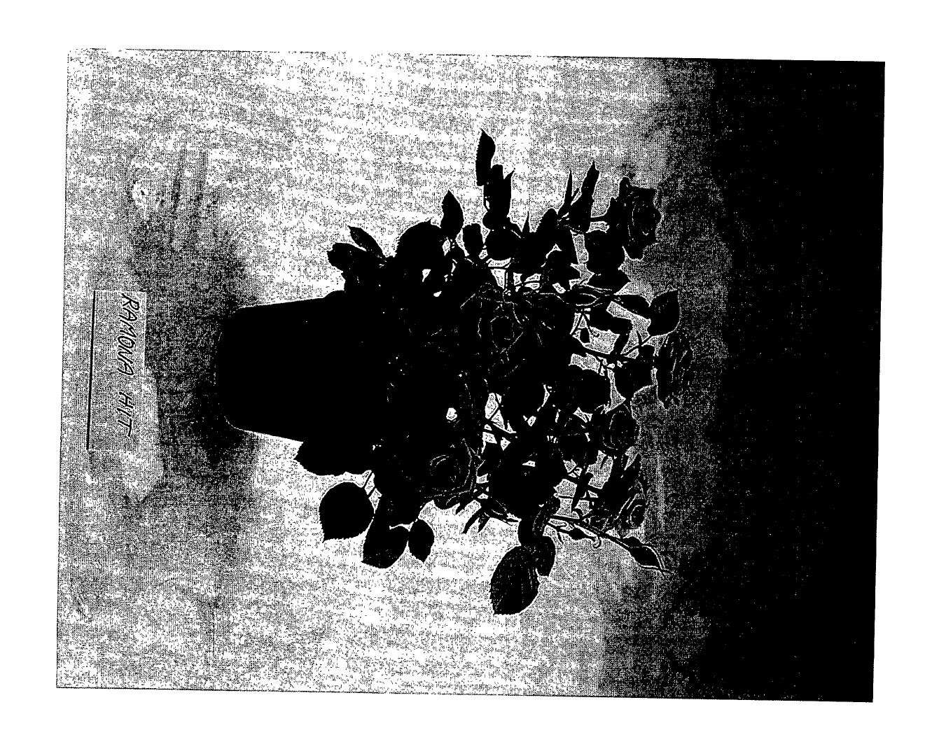

### SUMMARY OF THE INVENTION

The present discovery constitutes a new and distinct variety of a miniature pot rose plant which was discovered in a cultivated area. The mutation resulted from ‘POULsabel’, a miniature pot rose hybridized by the same inventors. ‘POULsabel’ is described and illustrated in U.S. Plant patent application Ser. No. 09/607,333, dated 30 Jun., 2000. The new rose variety resulted from a naturally occurring mutation of unknown causation on a branch of ‘POULsabel’.

The rose plant of the present discovery has a unique combination of characteristics which are outstanding in the new variety and which distinguish it from the original rose ‘POULsabel’ as well as all other varieties which we are aware of. For example, the new variety has:

1. Uniform and large flowers for a miniature rose;

2. Vigorous and compact growth;

3. Year-round flowering under glasshouse conditions;

4. Suitability for production from softwood cuttings in pots;

5. Durable flowers and foliage which make a variety suitable for distribution in the floral industry.

This combination of qualities is not present in previously available commercial cultivars of this type and distinguish ‘POULmona’ from all other varieties of which we are aware.

The resulting mutation was selected and evaluations were conducted on the resulting rose plants in a controlled environment.

Asexual reproduction of ‘POULmona’ by cuttings and traditional budding was first done by L. Pernille and Mogens N. Olesen in their nursery in Fredensborg, Denmark in June of 1998 at their nursery in Fredensborg, Denmark. This initial and other subsequent propagations conducted in controlled environments have demonstrated that the characteristics of ‘POULmona’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems of ‘POULmona’.

Specifically illustrated in SHEET 1 include ‘POULmona’s' foliage, flower buds, partially opened buds, and numerous open blooms.

Specifically illustrated in SHEET 2 are three blooms at different stages of opening.

### DETAILED DESCRIPTION OF THE VARIETY

The following is a description of ‘POULmona’, as observed in its growth in Half Moon Bay, Calif. Color references are made using The Royal Horticultural Society (London, England) Colour Chart, 1995, except where common terms of color are used.

For a comparison, several physical characteristics of the rose variety ‘POULsabel’, a rose variety from the same inventors described and illustrated in U.S. Plant patent application Ser. No. 09/607,333 , dated 30 Jun., 2000, are compared to ‘POULmona’ in Chart 1.

CHART 1

| | ‘POULmona’ | ‘POULsabel’ |
|---|---|---|
| Bud Color at | Red-Purple Group 58 | Red Group |
| ¼ open. | B and C. | 45A. |
| Upper Petal | Red-Purple Group | Red Group |
| Surface, Open | | 44B. |
| Bloom. | | |
| Petalage | 25 to 30 petals. | 30 to 35 |
| | | petals. |

| | |
|---|---|
| Parents: | Sport of Miniature Rose Variety |
| | ‘POULsabel’ |
| Classification: | Rosa Hybrida |
| Botanical: | Rosa hybrida. |
| Commercial: | Miniature. |

### FLOWER AND FLOWER BUD

| | |
|---|---|
| Blooming habit: | Recurrent/continuous. |
| Flower bud: | |
| Size: | Upon opening, 35 mm in |
| | length from base of |
| | receptacle to end of bud. |
| Bud form: | Pointed ovoid. |
| Bud color: | As sepals unfold, upper |
| | surface of sepals is Red- |
| | Purple Group 58 B and C; |
| | lower surface is Red-Purple |
| | Group 58 B and C. At ¼ |
| | opening, upper surface of |
| | sepals is Red-Purple Group |
| | 58 B and C; Red-Purple Group |
| | 58 B and C. |
| Sepals: | |
| Upper surface: | Yellow-Green Group |
| | 144A. |
| Lower surface: | Green Group 138B. |
| | Weak foliaceous |
| | appendages on |
| | three of the five |
| | sepals. Surfaces |
| | of sepals slightly |
| | pubescent. |
| | Stipitate glands |
| | present on sepal |
| | margins. |
| Shape: | Sepal apex is cirrose. |
| | Base is flat at union |
| | with peduncle. |
| Size: | 25 to 30 mm long × 10 |
| | to 12 mm wide. |
| Receptacle: | |
| Surface: | Smooth. |
| Shape: | Funnel shaped. |
| Size: | 8 mm (h) × 7 mm (w). |
| Color: | Yellow-Green Group |
| | 144A. |
| Peduncle: | |
| Surface: | Many fragrant stipitate |
| | glands present. |
| Length: | 40 to 50 mm average |
| | length. |
| Color: | Yellow-Green Group |
| | 144A. |
| Strength: | Strong. |
| Borne: | Singly. |

Flower bloom:

| | |
|---|---|
| Fragrance: | None. |
| Duration: | As a pot plant, flowers last |
| | from 5 to 7 days. |
| Size: | Medium for a 12 cm pot rose. |
| | Average flower diameter is |
| | 40 mm when open. |
| Form: | |
| Shape of flower when viewed | |
| from the side: | |
| Upon opening, upper part: | Cupped. |
| Upon opening, lower part: | Convex. |
| Open flower, upper part: | Convex. |
| Open flower, lower part: | Flattened |
| | convex. |
| Petalage: | Average range: 25 to 30 petals |
| | under normal conditions with 4 to |
| | 6 petaloids. |

Color:

| | |
|---|---|
| Upon opening, petals: | |
| Outermost petals: | |
| Outer Side: | Red-Purple Group |
| | 58B at margins, |
| | graduating to Red- |
| | Purple Group 58 C |
| | and D in middle |
| | zone. |
| Inner Side: | Red-Purple Group |
| | 58B at margins, |
| | graduating to Red- |
| | Purple Group 58 C |
| | and D in middle |
| | zone. |
| Innermost petals: | |
| Outer Side: | Red-Purple Group |
| | 58B at margins, |
| | graduating to Red- |
| | Purple Group 58 C |
| | and D in middle |
| | zone. |
| Inner Side: | Red-Purple Group |
| | 58B at margins, |
| | graduating to Red- |
| | Purple Group 58 C |
| | and D in middle |
| | zone. |
| Upon opening, basal petal spots: | |
| Outermost petals: | |
| Outer Side: | Green-Yellow Group |
| | 1C. |
| Inner Side: | Green-Yellow Group |
| | 1C. |
| Innermost petals: | |
| Outer Side: | Green-Yellow Group |
| | 1C. |
| Inner Side: | Green-Yellow Group |
| | 1C. |
| After opening, petals: | |
| Outermost petals: | |
| Outer Side: | Red-Purple Group |
| | 58C. |
| Inner Side: | Red-Purple Group |
| | 58C. |
| Innermost petals: | |
| Outer Side: | Red-Purple Group |
| | 58C. |
| Inner Side: | Red-Purple Group |
| | 58C. |
| After opening, basal petal spots: No | |
| distinctive coloration at petal base | |
| observed. | |
| Outermost petals: | |
| Outer Side: | Green-Yellow Group |
| | 1C. |
| Inner Side: | Green-Yellow Group |
| | 1C. |
| Innermost petals: | |
| Outer Side: | Green-Yellow Group |
| | 1C. |
| Inner Side: | Green-Yellow Group |
| | 1C. |
| General Tonality: | On open flower Red-Purple |
| | Group 58B. No change in the |
| | general tonality at the end |
| | of the 5th day. Afterwards, |
| | general tonality is Red- |
| | Purple Group 58C. |

Petals:

| | |
|---|---|
| Petal Reflex: | Petals reflex strongly. |
| Petal Edge: | Entire, with notch in |
| | center of margin. |
| Shape: | Deltoid. |
| Petaloids: | |
| Quantity: | 4 to 6. |
| Size: | 10 to 12 mm long; 4 to |
| | 6 mm wide. |
| Color: | Upper side is Red- |
| | Purple Group 58B and C. |
| | Lower side is Red- |
| | Purple Group 58 B. |
| Thickness: | Average. |
| Arrangement: | Imbricated. |

Reproductive organs:

| | |
|---|---|
| Pollen: | |
| Color: | Yellow-Orange Group |
| | 15A. |
| Quantity: | Scant. |
| Anthers: | |
| Size: | 2 mm long. |
| Color: | Yellow Group 11D. |
| Quantity: | Approximately 40. |
| Filaments: | |
| Color: | Yellow Group 10A. |
| Length: | 3 to 4 mm. |
| Stigmas: | Slightly superior in location to |
| | anthers. |
| Color: | Greyed-Yellow Group |
| | 160D. |
| Styles: | |
| Color: | Greyed-Yellow Group |
| | 160D, with intonations |
| | of Greyed-Green Group |
| | 193B. |
| Seed formation: | |
| Hips: | Not observed. |

PLANT

| | |
|---|---|
| Plant growth: | Vigorous, compact, upright to |
| | bushy. When grown as a 12 cm pot |
| | plant, the average height of the |
| | plant itself is 30 cm and the |
| | average width is 35 cm. |
| Stems: | |
| Color: | |
| Young wood: | yellow-Green Group |
| | 144B. |
| Older wood: | Yellow-Green Group |
| | 144A. |
| Prickles: | |
| Incidence: | 6 to 8 thorns per 10 cm |
| | of stem. |
| Size: | Average length: 4 mm. |
| Color: | Greyed-Red Group 181B. |
| Shape: | Linear. |
| Surface: | |
| Young wood: | Smooth. |
| Older wood: | Smooth. |
| Plant foliage: | Normal number of leaflets on |
| | normal leaves in middle of the |
| | stem: 5 leaflets. |
| Leaf size: | 90 mm (l) × 75 mm (w). |
| Color: | |
| Upper Leaf Surface: | Green Group 139A. |
| Lower Leaf Surface: | Green Group 139B. |
| Juvenile foliage: | |
| Upper Leaf Surface: | Yellow-Green Group |
| | 144A and B |
| Lower Leaf Surface: | Yellow-Green Group |
| | 144B. |
| Anthocyanin intonation: | |
| Location: | Margin of upper surface |
| | and all of lower |
| | surface exhibit |
| | anthocyanin coloration |
| | of Greyed-Red Group |
| | 181A. |
| Plant leaves and leaflets: | |
| Stipules: | |
| Size: | 4 mm. |
| Color: | Yellow-Green Group 145A. On |
| | plants grown under high |
| | light conditions, peduncle |
| | may exhibit intonations of |
| | Greyed-Red Group 181A. |
| Stipitate glands: | Present on edges of |
| | stipules. |
| Anthocyanin: | Greyed-Red Group 181A. |
| Petiole: | |
| Length: | 18 mm. |
| Color: | Yellow-Green Group 147A. |
| Underneath: | Yellow-Green |
| | 144A; one |
| | thorn |
| | present. |
| Margins: | Yellow-Green |
| | Group |
| | 144A. |
| Anthocyanin: | None noted. |
| Rachis: | |
| Color: | Green Group |
| Underneath: | Yellow-Green 144A; one |
| | thorn present. |
| Margins: | Yellow-Green Group |
| | 144A. |
| Anthocyanin: | |
| Leaflet: | |
| Edge: | Serrated. |
| Shape: | Ovate. |
| Texture: | Glossy and thick. |
| Arrangement: | Odd pinnate. |
| Venation: | Reticulate. |
| Disease resistance: | |

Average resistance to mildew, black spot, and
Botrytis under normal growing conditions in Jackson
County, Oregon.
Cold hardiness:
‘POULmona’ has been found to be resistant to
damage from cold in USDA Zone 7.

1. A new and distinct variety of rose of the miniature class, substantially as herein illustrated and described as a distinct and novel rose variety due to its abundant, red flowers, vigorous growth, compact habit, suitability for production from softwood cuttings in pots, and durable flowers and foliage which make the variety suitable for distribution in the floral industry.
